(19) United States
(12) Patent Application Publication
(10) Pub. No.: US 2023/0343439 A1
(43) Pub. Date: Oct. 26, 2023

(54) MEDICAL INFORMATION PROCESSING SYSTEM, MEDICAL INFORMATION PROCESSING METHOD, AND STORAGE MEDIUM

(71) Applicant: CANON MEDICAL SYSTEMS CORPORATION, Otawara-shi (JP)

(72) Inventors: Takuya SAKAGUCHI, Utsunomiya (JP); Yasuko FUJISAWA, Nasushiobara (JP); Chihiro HATTORI, Nasushiobara (JP); Kota AOYAGI, Nasushiobara (JP)

(73) Assignee: CANON MEDICAL SYSTEMS CORPORATION, Otawara-shi (JP)

(21) Appl. No.: 18/191,140

(22) Filed: Mar. 28, 2023

(30) Foreign Application Priority Data
Apr. 20, 2022 (JP) 2022-069365

(51) Int. Cl. G16H 30/40

(52) U.S. Cl. CPC G16H 30/40

(57) ABSTRACT

A medical information processing system of an embodiment includes processing circuitry. The processing circuitry acquires medical data regarding a first target of a patient according to an examination order. The processing circuitry generates first support information for supporting diagnosis of the first subject on the basis of the medical data. The processing circuitry analyzes a second subject different from the first subject on the basis of the medical data. The processing circuitry generates second support information for supporting diagnosis of the second target on the basis of an analysis result of the second target. The processing circuitry causes a display unit to display at least the second support information between the first support information and the second support information.

### CROSS-REFERENCE TO RELATED APPLICATION

The present application claims priority based on Japanese Patent Application No. 2022-069365, filed Apr. 20, 2022, the content of which is incorporated herein by reference.

### FIELD

Embodiments disclosed in this specification and drawings relate to a medical information processing system, a medical information processing method, and a storage medium.

### BACKGROUND

When an examination order is made based on an information system such as an electronic medical record system, if the examination is an image examination, a doctor will receive only one image, not a large number of images. In this case, it is important that the single image contains desired information. Currently, radiologists create images to be sent to electronic medical records based on visual observation and experience and send the images.

### BRIEF DESCRIPTION OF THE DRAWINGS

FIG. 1 is a diagram showing a configuration example of a medical information processing system in a first embodiment.

FIG. 2 is a diagram showing a configuration example of a medical information processing apparatus in the first embodiment,

FIG. 3 is a flowchart showing a flow of a series of processing of a processing circuitry according to the first embodiment.

FIG. 4 is a diagram showing an example of a display screen of an electronic medical record system.

FIG. 5 is a diagram showing another example of a display screen of the electronic medical record system.

FIG. 6 is a diagram showing a method of generating a non-order image.

FIG. 7 is a flowchart showing a flow of a series of processing of a processing circuitry according to a second embodiment.

FIG. 8 is a diagram showing a method of generating an order image.

FIG. 9 is a diagram showing an example of a medical knowledge base.

FIG. 10 is a flowchart showing a flow of a series of processing of a processing circuitry according to a third embodiment.

FIG. 11 is a diagram showing another example of the display screen of the electronic medical record system.

FIG. 12 is a diagram showing an image generation method when there are multiple destinations.

### DETAILED DESCRIPTION

Hereinafter, a medical information processing system, a medical information processing method, and a storage medium according to embodiments will be described with reference to the drawings. A medical information processing system of an embodiment includes processing circuitry. The processing circuitry acquires medical data regarding a first target of a patient according to an examination order. The processing circuitry generates first support information for supporting diagnosis of the first subject on the basis of the medical data. The processing circuitry analyzes a second subject different from the first subject on the basis of the medical data. The processing circuitry generates second support information for supporting diagnosis of the second target on the basis of an analysis result of the second target. The processing circuitry causes a display unit to display at least the second support information between the first support information and the second support information. With such a configuration, it is possible to automatically generate appropriate support information according to an examination order and return the support information to a user (orderer) that is an examination order source.

### First Embodiment

### [Configuration of Medical Information Processing System]

FIG. 1 is a diagram showing a configuration example of a medical information processing system 1 in a first embodiment. The medical information processing system 1 includes, for example, a medical information processing apparatus 100 and an information display apparatus 200. The medical information processing apparatus 100 and the information display apparatus 200 are connected via a communication network NW such that they can communicate with each other.

The communication network NW may mean any information communication network using telecommunication technology. For example, the communication network NW includes a wireless/wired LAN such as a hospital backbone local area network (LAN), the Internet network, a telephone communication network, an optical fiber communication network, a cable communication network, a satellite communication network, and the like.

The medical information processing apparatus 100 receives medical information from examination equipment via the communication network NW and processes the received medical information. The medical information processing apparatus 100 then transmits the processed information to the information display apparatus 200 via the communication network NW.

Examination equipment is equipment for medically examining a patient and includes, for example, an X-ray computed tomography (CT) apparatus, a magnetic resonance imaging (MRI) apparatus, a mammography apparatus, an ultrasonic imaging apparatus, a nuclear medicine diagnostic apparatus, a body fluid analysis apparatus, a device for measuring vital signs, etc.

The medical information processing apparatus 100 may be a single apparatus, or may be a system in which a plurality of apparatuses connected via the communication network NW operate in cooperation. That is, the medical information processing apparatus 100 may be realized by multiple computers (processors) included in a distributed computing system or a cloud computing system. Further, the medical information processing apparatus 100 does not necessarily have to be a separate apparatus different from the information display apparatus 200 and may be an apparatus integrated with the information display apparatus 200.

The information display apparatus 200 is, for example, an apparatus or system capable of recording and displaying patient medical data obtained by various types of examination equipment, and is typically an electronic medical record (EMR), electronic health record (EHR), personal health record (PHR), or the like. For example, the information display apparatus 200 includes a liquid crystal display (LCD), a cathode ray tube (CRT) display, an organic electroluminescence (EL) display, and the like. In addition, the information display apparatus 200 is not limited to a dedicated apparatus or system for recording information on patient health and medical care, and may be a general-purpose terminal device such as a smartphone or a tablet, or a display in which an image viewer is installed. Hereinafter, as an example, the information display apparatus 200 will be described as an EMR, that is, an electronic medical record system. The information display apparatus 200 is an example of a “display unit.”

### [Configuration of Medical Information Processing Apparatus]

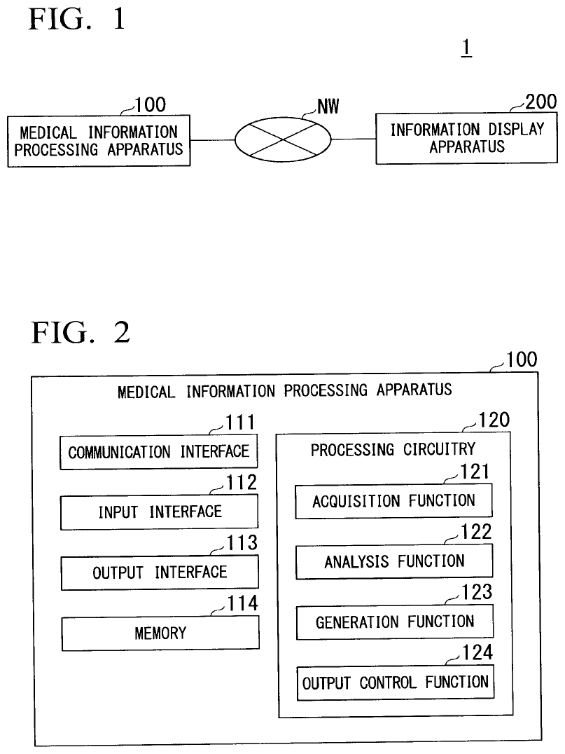

FIG. 2 is a diagram showing a configuration example of the medical information processing apparatus 100 in the first embodiment. The medical information processing apparatus 100 includes, for example, a communication interface 111, an input interface 112, an output interface 113, a memory 114, and a processing circuitry 120.

The communication interface 111 communicates with the information display apparatus 200, examination equipment, etc. via the communication network NW. The communication interface 111 includes, for example, a network interface card (NIC), an antenna for wireless communication, and the like.

The input interface 112 receives various input operations from an operator, converts the received input operations into electrical signals, and outputs the electrical signals to the processing circuitry 120. For example, the input interface 112 includes a mouse, a keyboard, a trackball, a switch, a button, a joystick, a touch panel, and the like. The input interface 112 may be, for example, a user interface that receives voice input, such as a microphone. When the input interface 112 is a touch panel, the input interface 112 may also have a display function of a display 113a included in the output interface 113, which will be described later.

The input interface 112 in this specification is not limited to one having physical operation components such as a mouse and a keyboard. For example, examples of the input interface 112 also include an electrical signal processing circuit that receives an electrical signal corresponding to an input operation from external input equipment provided separately from the apparatus and outputs the electrical signal to a control circuit.

The output interface 113 includes, for example, the display 113a, a speaker 113b, and the like.

The display 113a displays various types of information. For example, the display 113a displays an image generated by the processing circuitry 120, a graphical user interface (GUI) for receiving various input operations from the operator, and the like. For example, the display 113a is an LCD, a CRT display, an organic EL display, or the like.

The speaker 113b outputs information input from the processing circuitry 120 as sound.

The memory 114 is realized by, for example, a semiconductor memory element such as a random-access memory (RAM) or a flash memory, a hard disk, or an optical disc. These non-transitory storage media may be realized by other storage devices such as a network attached storage (NAS) and external storage server devices connected via the communication network NW. Further, the memory 114 may also include non-transitory storage media such as a read only memory (ROM) and a register.

The memory 114 stores model information in addition to programs executed by a hardware processor. The model information is information (program or data structure) that defines a machine learning model MDL, which will be described later. The machine learning model MDL may be implemented by, for example, deep neural network(s) (DNNs) such as a convolutional neural network (CNN). Further, the machine learning model MDL is not limited to DNNs and may be implemented by other models such as a support vector machine, a decision tree, a naive Bayes classifier, and a random forest. Details of the machine learning model MDL will be described later.

When the machine learning model MDL is implemented by a DNN, the model information includes, for example, coupling information representing how units included in each of an input layer, one or more hidden layers (intermediate layers), and an output layer that constitute the DNN are coupled to each other, weight information representing how many coupling coefficients are provided to data input/output between coupled units, and the like. The coupling information includes, for example, information such as the number of units included in each layer, information for designating the type of a unit that is a coupling destination of each unit, an activation function that realizes each unit, and a gate provided between units in the hidden layers. The activation function that realizes a unit may be, for example, a rectified linear unit (ReLU) function, an exponential linear units (ELU) function, a clipping function, a sigmoid function, a step function, a hyperbolic tangent function, an identity function, or the like. The gate selectively passes or weights data transmitted between units, for example, depending on a value (e.g., 1 or 0) returned by the activation function. A coupling coefficient includes, for example, a weight applied to output data when data is output from a unit in a certain layer to a unit in a deeper layer in a hidden layer of the neural network. Further, the coupling coefficients may also include a bias component unique to each layer, and the like.

The processing circuitry 120 includes, for example, an acquisition function 121, an analysis function 122, a generation function 123, and an output control function 124. The acquisition function 121 is an example of an “acquisition unit,” the analysis function 122 is an example of an “analysis unit,” and the generation function 123 is an example of a “generation unit,” a “first generation unit”, and/or a “second generation unit.” The output control function 124 is an example of a “display control unit.”

The processing circuitry 120 realizes these functions by a hardware processor (computer) executing a program stored in the memory 114 (storage circuit), for example.

The hardware processor in the processing circuitry 120 is, for example, a circuit (circuitry) such as a central processing unit (CPU), a graphics processing unit (GPU), an application specific integrated circuit (ASIC), and a programmable logic device (for example, a simple programmable logic device (SPLD), a complex programmable logic device (CPLD), or a field programmable gate array (FPGA)). Instead of storing the program in the memory 114, the program may be directly incorporated into the circuit of the hardware processor. In this case, the hardware processor realizes the functions by reading and executing the program incorporated into the circuit. The aforementioned program may be stored in the memory 114 in advance, or may be stored in a non-transient storage medium such as a DVD or a CD-ROM and installed in the memory 114 from the non-transient storage medium by setting the non-transient storage medium in a drive device (not shown) of the medical information processing apparatus 100. The hardware processor is not limited to being configured as a single circuit, and may be configured as one hardware processor by combining a plurality of independent circuits to realize each function. Further, a plurality of components may be integrated into one hardware processor to realize each function.

### [Processing Flow of Medical Information Processing Apparatus]

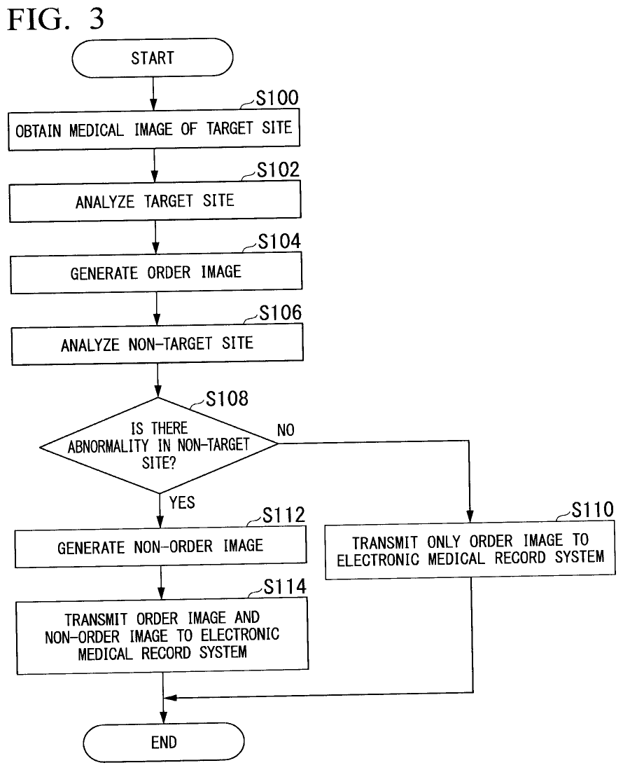

Hereinafter, a series of processing performed by the processing circuitry 120 of the medical information processing apparatus 100 will be described with reference to a flowchart. FIG. 3 is a flowchart showing a flow of a series of processing of the processing circuitry 120 according to the first embodiment.

Processing of this flowchart may be executed, for example, when an orderer such as a doctor places an examination order. An examination order is information for requesting that the medical information processing apparatus 100 examine a target site suspected of having a disease using patient medical data (CT images, MR images, ultrasound images, PET images, blood data, gene data, etc.) examined by various types of examination equipment such as an X-ray CT apparatus, an MRI apparatus, a mammography apparatus, an ultrasonic imaging apparatus, a nuclear medicine diagnostic apparatus, and a body fluid analysis apparatus. An examination order may be read as an examination request, an examination query, or the like.

For example, examination equipment that has completed an examination may transmit an examination order along with medical data to the medical information processing apparatus 100 via the communication network NW. Further, the orderer may input medical data and an examination order to the input interface 112 of the medical information processing apparatus 100. In the following description, as an example, medical data is a medical image such as a CT image or an MR image.

First, the acquisition function 121 acquires a medical image of a target site from the examination equipment via the communication interface 111 or acquires a medical image of the target site via the input interface 112 (step S100). In the following description, as an example, the target site is the “liver.” That is, the acquisition function 121 acquires a medical image of the liver. The liver is an example of a “first target.”

Next, the analysis function 122 analyzes the liver, which is the target site specified by the examination order, on the basis of the medical image acquired by the acquisition function 121 (step S102).

For example, the analysis function 122 may extract liver features from the medical image by performing image processing on the medical image and determine whether or not there is an abnormality in the liver on the basis of the extracted features. Abnormalities in the liver include, for example, occurrence of diseases such as fatty liver, constitutional jaundice, viral hepatitis, and liver cancer.

Further, the analysis function 122 may determine the presence or absence of an abnormality in the liver and probability thereof from the medical image using a machine learning model MDL defined by model information in the memory 114.

The machine learning model MDL is machine learning trained using, as training data, a data set in which, for medical images of a liver that has already suffered from a disease or is highly likely to have a disease, the disease is labeled as correct answer information. In other words, the machine learning model MDL is a machine learning model trained to output a probability indicating a likelihood of a disease occurring in a liver when a certain liver medical image is input thereto.

When a medical image of the liver of a certain patient is input to the machine learning model MDL trained using such training data, the machine learning model MDL outputs the probability of a disease that can occur in the liver of the patient as an estimation result. An estimation result of the machine learning model MDL is represented by, for example, a multidimensional vector or tensor. The vector or tensor includes the likelihood (probability) of having a disease as an element value. For example, it is assumed that there are four types of liver diseases: fatty liver, constitutional jaundice, viral hepatitis, and liver cancer. In this case, the vector or tensor can be represented as (e1, e2, e3, e4) when the probability of fatty liver is e1, the probability of constitutional jaundice is e2, the probability of viral hepatitis is e3, and the probability of liver cancer is e4.

The machine learning model MDL may be trained for general use using a training data set of other sites in addition to the liver. That is, a single machine learning model MDL may be trained to estimate a disease of each of a plurality of sites. Further, the machine learning model MDL may be individually prepared for each site. For example, a dedicated machine learning model MDL for estimating liver diseases, a dedicated machine learning model MDL for estimating pancreatic diseases, a dedicated machine learning model MDL for estimating kidney diseases, and the like may be prepared.

Further, the analysis function 122 may determine the presence or absence of an abnormality in the liver and the probability thereof on the basis of a rule base in addition to or instead of a machine learning base.

Next, the generation function 123 generates a medical image (hereinafter referred to as an order image IMG1) according to the examination order (step S104).

The order image IMG1 is an image in which the liver, which is the target site designated by the examination order, is easily visually recognized. For example, when there is a disease in the liver, the generation function 123 may select a cross-sectional image in which the disease is most conspicuous from among a plurality of medical images of the liver acquired as medical data at the time of receiving the examination order and generate the selected cross-sectional image as the order image IMG1. The order image IMG1 is an example of “first support information.”

Next, the analysis function 122 analyzes another site (hereinafter referred to as a non-target site) different from the target site on the basis of the medical image of the liver acquired by the acquisition function 121 (step S106).

For example, if the target site is the liver, a non-target site may be the pancreas, which is likely to be present in the medical image of the liver, or the like. That is, a non-target site is a site present in the body at a position close to the target site. In the following description, as an example, a non-target site is the pancreas.

For example, the analysis function 122 may extract features of the pancreas from the medical image by performing image processing on the medical image on which liver analysis has been performed and determine whether or not there is an abnormality in the pancreas on the basis of the extracted features. Abnormalities in the pancreas include, for example, occurrence of diseases such as pancreatitis, pancreatic cyst, and pancreatic cancer.

Further, the analysis function 122 may determine the presence or absence of an abnormality in the pancreas and the probability thereof from the medical image using the machine learning model MDL defined by the model information in the memory 114.

Further, the analysis function 122 may determine the presence or absence of an abnormality in the pancreas and the probability thereof on the basis of a rule base in addition to or instead of a machine learning base.

Next, the generation function 123 determines whether or not there is an abnormality in the pancreas on the basis of an analysis result of the pancreas, which is a non-target site (step S108).

If there is no abnormality in the pancreas, the output control function 124 transmits only the order image IMG1 to the electronic medical record system 200 via the communication interface 111 (step S110). The electronic medical record system 200 receives the order image IMG1 and displays it as part of the electronic medical record.

On the other hand, if there is an abnormality in the pancreas, the generation function 123 generates a non-order image IMG2 (step S112). The non-order image IMG2 is a medical image in which the pancreas, which is a non-target site not specified by the examination order, is easily visually recognized. The non-order image IMG2 is an example of “second support information.”

For example, the generation function 123 may generate a two-dimensional image such as VR, MPR, CPR, SPR, MIP, MinIP, or SlabMIT as the non-order image IMG2 on the basis of the features of the pancreas.

At this time, the generation function 123 may generate the non-order image IMG2 on the basis of data capacity restriction at the time of causing the electronic medical record system 200 to display an image. In general, images handled by the electronic medical record system 200 are limited in size and data amount. Therefore, the generation function 123 generates the non-order image IMG2 having a required minimum image quality and size such that constraints on the data capacity handled by the electronic medical record system 200 are satisfied.

For example, the generation function 123 may generate, as the non-order image a medical image having a size that includes the entire region of the pancreas determined to be abnormal among a plurality of medical images acquired as medical data at the time of receiving an examination order. Moreover, the generation function 123 may generate, as the non-order image IMG2, a medical image having a direction or slice including the entire region of the pancreas determined to be abnormal among the plurality of medical images. Further, when a plurality of abnormal positions are present in the pancreas, the generation function 123 may generate, as the non-order image IMG2, a medical image including all of the plurality of positions. In addition, when a plurality of abnormal positions are present in the pancreas, the generation function 123 may generate a medical image of a slice including a position having the most conspicuous abnormality among the plurality of positions as the non-order image IMG2 or generate a medical image at a time including the position having the most conspicuous abnormality as the non-order image IMG2. Further, when a plurality of abnormal positions are present in the pancreas, the generation function 123 may generate, as the non-order image IMG2, an enlarged image of a position having the most conspicuous abnormality among the plurality of positions. Further, the generation function 123 may generate, as the non-order image IMG2, a medical image in which the contrast of the position having the most conspicuous abnormality is a maximum density. In addition, the generation function 123 may generate, as the non-order image IMG2, a medical image having a direction or angle in/at which a position having the most conspicuous abnormality appears to be longest, or an angle at which bifurcations can be separated widest. Further, when there a plurality of abnormal positions are present in the pancreas, the generation function 123 may generate a medical image in which the plurality of positions do not overlap as the non-order image IMG2.

Furthermore, the generation function 123 may superimpose an image object (for example, an arrow, color, fill, an outline, characters, blinking, or the like) on the non-order image IMG2 to draw attention to an abnormal position.

Furthermore, the generation function 123 may generate, as the non-order image IMG2, a medical image of the pancreas with an orientation changed such that the orientation of the pancreas is the same as the typical direction of the pancreas in medical textbooks. Accordingly, even if the pancreas for which an examination order has not been placed is displayed, the doctor can easily understand a disease of the pancreas without being surprised.

Next, when there is an abnormality in the pancreas and the non-order image IMG2 is generated, the output control function 124 transmits the order image IMG1 and the non-order image IMG2 to the electronic medical record system 200 via the communication interface 111 (Step S114). The electronic medical record system 200 receives the order image IMG1 and the non-order image IMG2 and displays them as part of the electronic medical record. Accordingly, processing of this flowchart ends.

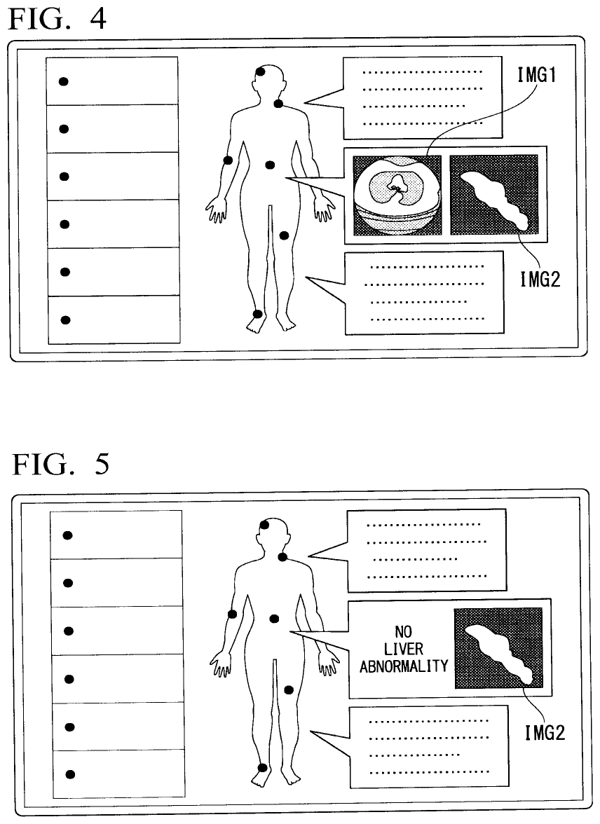

FIG. 4 is a diagram showing an example of a display screen of the electronic medical record system 200. When the electronic medical record system 200 receives the order image IMG1 and the non-order image IMG2 from medical information processing apparatus 100, the electronic medical record system 200 may display the order image IMG1 and the non-order image IMG2 side by side as illustrated.

FIG. 5 is a diagram showing another example of the display screen of the electronic medical record system 200. For example, when there is no abnormality in the liver and there is an abnormality in the pancreas, the medical information processing apparatus 100 may transmit information indicating that there is no abnormality in the liver instead of the order image IMG1, and the non-order image IMG2 to the electronic medical record system 200. In this case, the electronic medical record system 200 may display a message representing that there is no abnormality in the liver instead of the order image IMG1, and the non-order image IMG2 side by side.

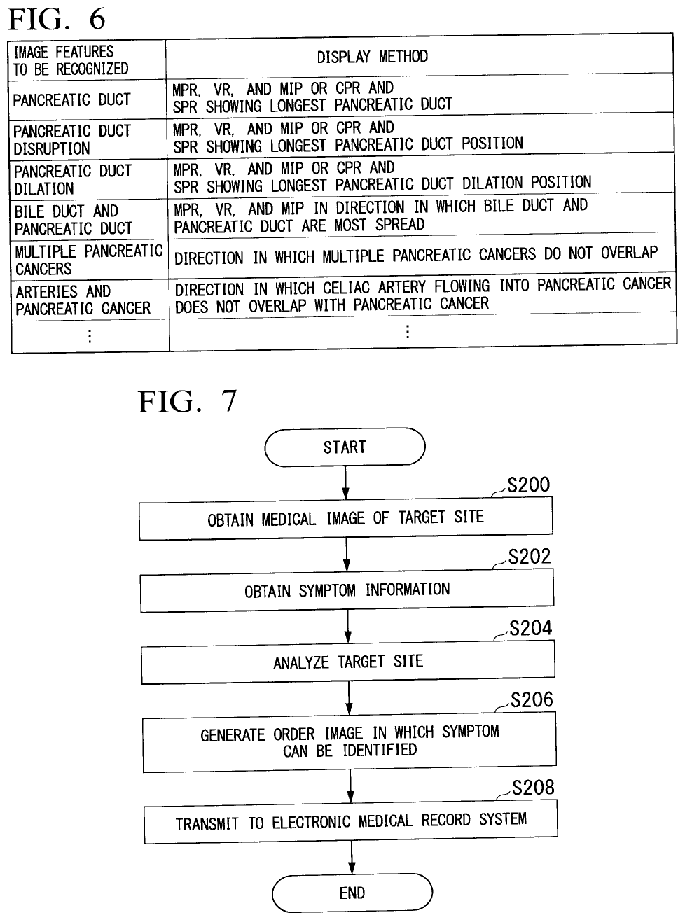

FIG. 6 is a diagram showing a method of generating the non-order image IMG2. For example, the generation function 123 may generate the non-order image IMG2 such that a display mode is changed according to characteristics of the pancreas determined to be abnormal.

As shown in the figure, for example, when it is recognized that an abnormality has occurred in the pancreatic duct, the generation function 123 may generate MPR, VR, MIP, CPR, and SPR in which the pancreatic duct appears the longest as the non-order image IMG2.

According to the first embodiment described above, the processing circuitry 120 of the medical information processing apparatus 100 acquires a medical image (an example of “medical data”) regarding the liver of a patient according to an examination order. The processing circuitry 120 generates an order image IMG1 (an example of “first support information”) for supporting diagnosis of the liver on the basis of the medical image of the liver. On the other hand, the processing circuitry 120 analyzes the pancreas, which is different from the liver designated by the examination order, on the basis of the medical image of the liver. The processing circuitry 120 generates a non-order image IMG2 (an example of “second support information”) for supporting diagnosis of the pancreas on the basis of an analysis result of the pancreas. Then, the processing circuitry 120 causes the electronic medical record system 200 to display at least the non-order image IMG2 between the order image IMG1 and the non-order image IMG2. Accordingly, it is possible to return support information for the pancreas, additionally analyzed according to a liver examination order, to a doctor who has placed the liver examination order.

(Modified Example of First Embodiment)

Hereinafter, a modified example of the first embodiment will be described. In the modified example of the first embodiment, when a liver disease to be analyzed is designated by an examination order, if another disease different from the disease is found on an image of the liver, a medical image in which the other disease is easily visually recognized may be generated as a non-order image IMG2.

For example, it is assumed that “liver cancer” is designated by a liver examination order. In this case, the analysis function 122 analyzes whether “liver cancer” has occurred in the liver on the basis of a medical image of the liver, and also analyzes whether other diseases that can occur in the liver, for example, “viral hepatitis,” have occurred. Liver cancer is an example of a “first disease” and viral hepatitis is an example of a “second disease.”

When it is determined that “viral hepatitis” has occurred in the liver, the generation function 123 generates a medical image in which the presence or absence of “liver cancer” can be easily visually recognized as an order image IMG1 and generates a medical image in which occurrence of “viral hepatitis” can be easily visually recognized as a non-order image IMG2. Then, the output control function 124 transmits the order image IMG1 and the non-order image IMG2 to the electronic medical record system 200 via the communication interface 111.

Further, in the modified example of the first embodiment, when an additional disease is found in an examination other than an image (for example, a blood test or the like), the position of the disease may be superimposed on a representative medical image. Further, in the modified example of the first embodiment, instead of or in addition to displaying the order image IMG1 and the non-order image IMG2 on the electronic medical record system 200, the order image IMG1 and the non-order image IMG2 may be displayed on the display 113a of the medical information processing apparatus 100.

### Second Embodiment

Hereinafter, a second embodiment will be described. The second embodiment differs from the first embodiment in that the medical information processing apparatus 100 acquires not only medical images but also auxiliary information at the time of receiving an examination order and generates an order image IMG1 suitable for symptoms of a patient on the basis of the auxiliary information. The auxiliary information includes information indicating symptoms of the patient (hereinafter referred to as symptom information), information indicating the type of examination, and the like. Hereinafter, differences from the first embodiment will be mainly described and description common to the first embodiment will be omitted. In addition, in the description of the second embodiment, the same part as those of the first embodiment are denoted by the same reference numerals.

FIG. 7 is a flowchart showing a flow of a series of processing of the processing circuitry 120 according to the second embodiment.

First, the acquisition function 121 acquires a medical image of the liver, which is a target site, from examination equipment via the communication interface 111 or acquires a medical image of the liver via the input interface 112 (step S200).

Further, the acquisition function 121 acquires symptom information (step S202).

Next, the analysis function 122 analyzes the liver, which is the target site designated by an examination order, on the basis of the medical image acquired by the acquisition function 121 (step S204).

Next, the generation function 123 generates an order image IMG1 capable of identifying a symptom designated by the examination order (step S206). In other words, the generation function 123 generates a medical image in which the symptom designated by the examination order most conspicuously appear as the order image IMG1.

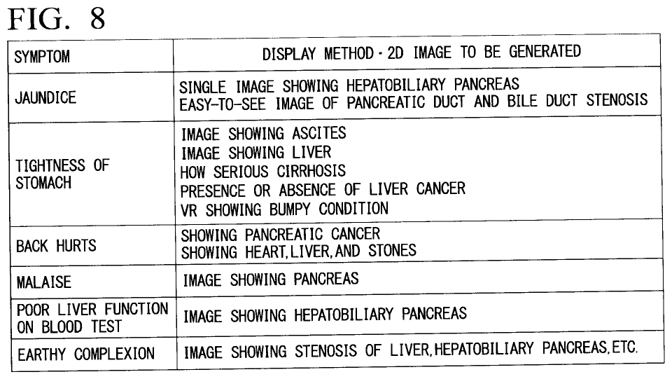

FIG. 8 is a diagram showing a method of generating the order image IMG1. For example, the generation function 123 may generate the order image IMG1 in which the symptom designated by the examination order can be identified using a table in which a mode of an image to be generated (to be displayed) is associated with each symptom, as shown in FIG. 8.

For example, if the symptom designated by the examination order is “jaundice,” the generation function 123 may generate an image showing the hepatobiliary pancreas as the order image IMG1 or generate an image in which pancreatic duct stenosis and bile duct stenosis are easily viewed as the order image IMG1.

The generation function 123 may also generate the order image IMG1 in which the symptom designated by the examination order can be identified using a medical knowledge base.

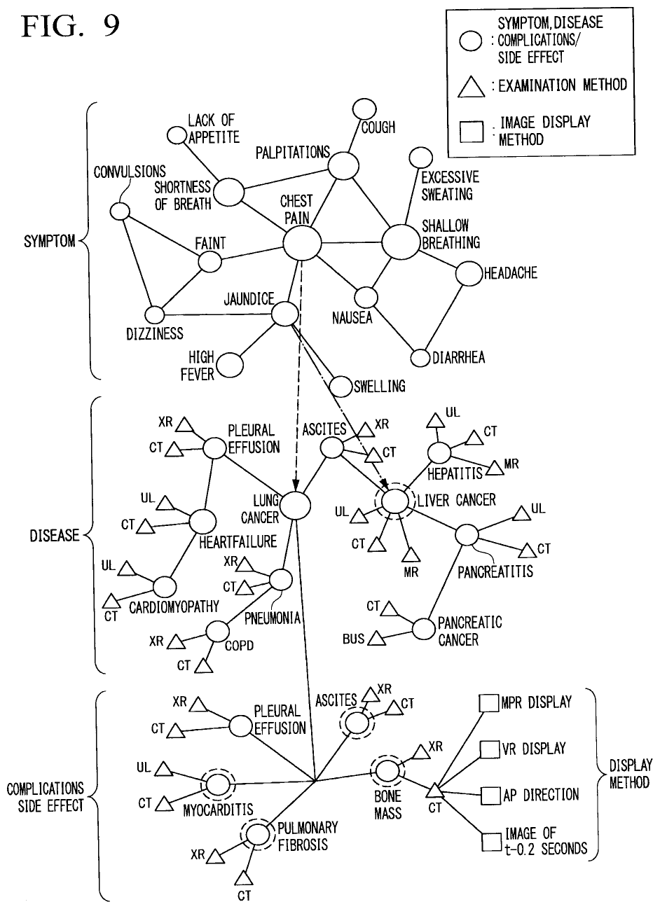

FIG. 9 is a diagram showing an example of a medical knowledge base. As illustrated, the medical knowledge base may be, for example, a database in which symptoms, diseases estimated from the symptoms, examinations corresponding to the diseases, and display modes of medical images obtained by the examinations are formalized by a conceptual system called ontology. An ontology defines classes and properties of nodes (also called entities) and collects constraints established between classes and properties.

For example, the generation function 123 searches for a symptom designated by an examination order on the medical knowledge base. The generation function 123 estimates a disease from the symptom found by the search, estimates a necessary examination from the estimated disease, and further estimates a display mode of medical images from the examination method. Then, the generation function 123 generates an order image IMG1 that matches the display mode of medical images.

Return to the description of the flowchart. Next, the output control function 124 transmits the order image IMG1 to the electronic medical record system 200 via the communication interface 111 (step S208). The electronic medical record system 200 receives the order image IMG1 and displays it as part of the electronic medical record. Accordingly, processing of this flowchart ends.

In the second embodiment, the order image IMG1 may be changed according to circumstances of each hospital or may be changed according to the presence or absence of a staff. Furthermore, the order image IMG1 may be changed between night and day, or the order image IMG1 may be changed according to the age of the patient.

According to the second embodiment described above, the processing circuitry 120 acquires auxiliary information including a symptom of a patient designated by an examination order and generates an order image IMG1 according to the symptom of the patient. Accordingly, it is possible to return more appropriate support information to a doctor who has placed the examination order.

### Third Embodiment

Hereinafter, a third embodiment will be described. The third embodiment differs from the above-described embodiments in that, when it is analyzed that a disease has occurred in the pancreas, which is a non-target site, the electronic medical record system 200 is caused to display a medical image of the diseased pancreas as a non-order image IMG2 and to display a medical image of the pancreas, which was free of disease when analyzed in the past, as a second non-order image IMG2 #. In the following, differences from the first embodiment and the second embodiment will be mainly described, and description of points common to the first embodiment and the second embodiment will be omitted. In addition, in the description of the third embodiment, the same parts as those in the first embodiment or the second embodiment are denoted by the same reference numerals.

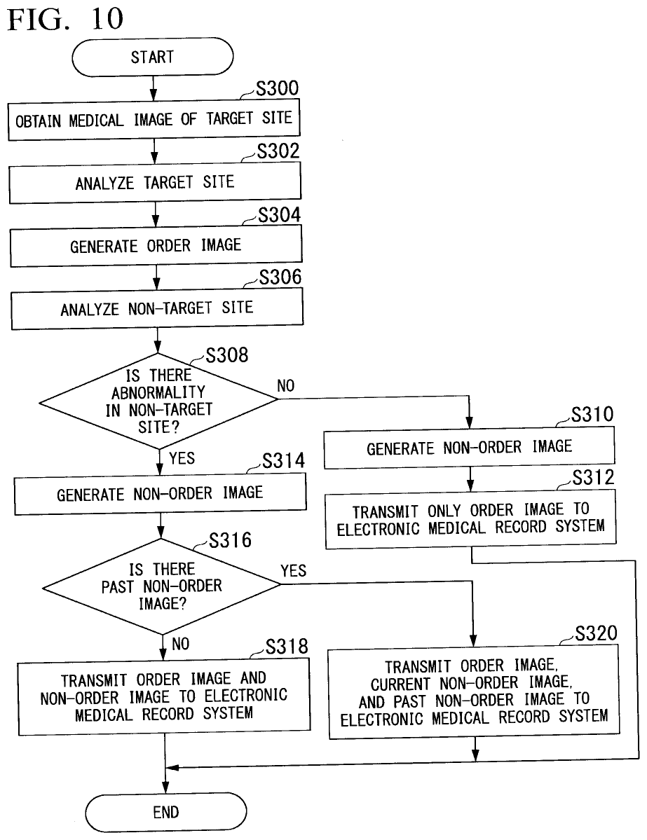

FIG. 10 is a flowchart showing a flow of a series of processing of the processing circuitry 120 according to the third embodiment.

First, the acquisition function 121 acquires a medical image of the liver, which is a target site, from examination equipment via the communication interface 111 or acquires a medical image of the liver via the input interface 112 (step S300).

Next, the analysis function 122 analyzes the liver, which is the target site designated by an examination order, on the basis of the medical image acquired by the acquisition function 121 (step S302).

Next, the generation function 123 generates an order image IMG1 of the liver as a medical image according to the examination order (step S304).

Next, the analysis function 122 analyzes the pancreas, which is a non-target site, on the basis of the medical image of the liver acquired by the acquisition function 121 (step S306).

Next, the generation function 123 determines whether or not there is an abnormality in the pancreas on the basis of an analysis result of the pancreas, which is a non-target site (step S308).

If the pancreas does not have an abnormality, the generation function 123 generates a non-order image IMG2 of the pancreas and stores it in the memory 114 (step S310).

Next, the output control function 124 transmits only the order image IMG1 to the electronic medical record system 200 via the communication interface 111 (step S312). The electronic medical record system 200 receives the order image IMG1 and displays it as part of the electronic medical record.

On the other hand, if an abnormality has occurred in the pancreas, the generation function 123 generates a non-order image IMG2 of the pancreas (step S314).

Next, the output control function 124 determines whether or not a non-order image IMG2 of the pancreas which was generated in past processing even though there was no abnormality in the pancreas, that is, a non-order image IMG2 # of the pancreas which was generated in the past, is present (is stored) in the memory 114 (step S316).

The output control function 124 transmits the order image IMG1 and the currently generated non-order image IMG2 to the electronic medical record system 200 via the communication interface 111 when the non-order image IMG2 # of the pancreas having no abnormality is not present (is not stored) in the memory 114 (step S318). The electronic medical record system 200 receives the order image IMG1 and the currently generated non-order image IMG2 and displays them as part of the electronic medical record.

On the other hand, if the non-order image IMG2 # of the pancreas having no abnormality is present (is stored) in the memory 114, the output control function 124 transmits the order image IMG1, the currently generated non-order image IMG2, and the non-order image IMG2 # generated in the past to the electronic medical record system 200 via the communication interface 111 (step S320). The electronic medical record system 200 receives the order image IMG1, the currently generated non-order image IMG2, and the non-order image IMG2 # generated in the past and displays them as part of the electronic medical record.

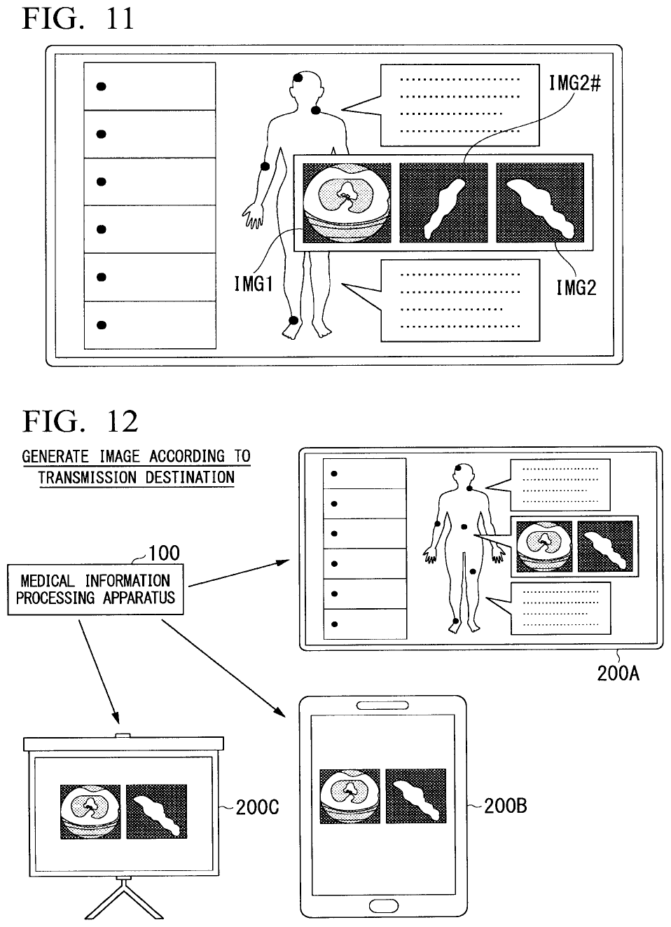

FIG. 11 is a diagram showing another example of the display screen of the electronic medical record system 200. For example, if there is an abnormality in the pancreas at the time of the current examination order, the medical information processing apparatus 100 transmits a non-order image IMG2 # generated although there was no abnormality at the time of a past examination order, and a non-order image IMG2 generated at the time of the current examination order to the electronic medical record system 200. In this case, the electronic medical record system 200 may display the non-order image IMG2 of the pancreas in which an abnormality has been found at the time of the current examination order and the non-order image IMG2 # of the pancreas in which no abnormality was found at the time of the previous examination order side by side.

According to the third embodiment described above, the processing circuitry 120 causes the electronic medical record system 200 to display a non-order image IMG2 of the pancreas in which an abnormality has been found at the time of the current examination order and a non-order image IMG2 # of the pancreas in which no abnormality was found in the previous examination order. Accordingly, it is possible to return more appropriate support information to a doctor who has placed the examination orders.

### OTHER EMBODIMENTS

Other embodiments will be described below. Although a transmission destination of the order image IMG1 and the non-order image IMG2 has been described as the electronic medical record system in the above-described embodiments, the present invention is not limited thereto. For example, if there are a plurality of transmission destinations of the order image IMG1 and the non-order image IMG2, the processing circuitry 120 may generate images suitable for each transmission destination device or system.

FIG. 12 is a diagram showing an image generation method when there are a plurality of transmission destinations. In the illustrated example, an electronic medical record system 200A, a patient's smartphone 200B, and a display 200C in which an image viewer is installed are present. In such a case, the processing circuitry 120 generates image for the electronic medical record system 200A such that constraints of data capacity handled by the electronic medical record system 200A are satisfied. Similarly, the processing circuitry 120 generates images for the patient's smartphone 200B such that constraints of data capacity handled by the smartphone 200B are satisfied. Similarly, the processing circuitry 120 generates images for the display 200C such that constraints of data capacity handled by display 200C are satisfied.

Further, although the medical information processing apparatus 100 and the information display apparatus (electronic medical record system) 200 are different apparatuses in the above-described embodiments, the present invention is not limited thereto. For example, the medical information processing apparatus 100 and the information display apparatus 200 may be integrated into one apparatus.

Although several embodiments have been described, these embodiments are presented as examples and are not intended to limit the scope of the invention. These embodiments can be implemented in various other forms, and various omissions, substitutions, and modifications can be made without departing from the spirit of the invention. These embodiments and modifications thereof are included in the scope and spirit of the invention, as well as the scope of the invention described in the claims and equivalents thereof.

What is claimed is:

1. A medical information processing system comprising processing circuitry configured to:
obtain medical data regarding a first target of a patient according to an examination order;
generate first support information for supporting diagnosis of the first target on the basis of the medical data;
analyze a second target different from the first target on the basis of the medical data;
generate second support information for supporting diagnosis of the second target on the basis of an analysis result of the second target; and
cause a display unit to display at least the second support information between the first support information and the second support information.

2. The medical information processing system according to claim 1, wherein the processing circuitry generates the second support information on the basis of restrictions on data capacity at the time of causing the display unit to display information.

3. The medical information processing system according to claim 1, wherein the second support information is a medical image of the second target, and
the processing circuitry causes the display unit to display at least the medical image of the second target as part of an electronic medical record.

4. The medical information processing system according to claim 3, wherein the medical data is a medical image of the first target, and
the processing circuitry analyzes the second target on the medical image of the first target and generates a medical image of the second target according to a state of the second target analyzed on the medical image of the first target.

5. The medical information processing system according to claim 1, wherein the first target is a first disease,
the second target is a second disease that is able to occur in the same site as the first disease, and
the processing circuitry generates the second support information for supporting diagnosis of the second disease when it is analyzed that the second disease has occurred.

6. The medical information processing system according to claim 1, wherein the processing circuitry further acquires auxiliary information including symptoms of the patient and generates the first support information according to the symptoms at the time of generating the first support information on the basis of the analysis result of the first target.

7. The medical information processing system according to claim 6, wherein the processing circuitry generates the first support information according to symptoms using a database in which symptoms, diseases estimated from the symptoms, examinations according to the diseases, and display modes of medical images obtained by the examinations are systematized by ontology.

8. The medical information processing system according to claim 4, wherein, when it is analyzed that the second target has a disease, the processing circuitry causes the display unit to display a medical image of the second target having the disease and a medical image of the second target analyzed as disease-free in the second target in the past side by side.

9. A medical information processing system comprising processing circuitry configured to:
acquire medical data regarding a first target of a patient and auxiliary information including symptoms of the patient according to an examination order;
analyze the first target on the basis of the medical data and the auxiliary information; and
generate first support information for supporting diagnosis of the first target on the basis of an analysis result of the first target; and
cause a display unit to display the first support information.

10. A medical information processing method executed using a computer, comprising:
obtaining medical data regarding a first target of a patient according to an examination order;
generating first support information for supporting diagnosis of the first target on the basis of the medical data;
analyzing a second target different from the first target on the basis of the medical data;
generating second support information for supporting diagnosis of the second target on the basis of an analysis result of the second target; and
causing a display unit to display at least the second support information between the first support information and the second support information.

11. A non-transitory computer-readable storage medium storing a program for being executed by a computer, the program comprising:
obtaining medical data regarding a first target of a patient according to an examination order;
generating first support information for supporting diagnosis of the first target on the basis of the medical data;
analyzing a second target different from the first target on the basis of the medical data;
generating second support information for supporting diagnosis of the second target on the basis of an analysis result of the second target; and
causing a display unit to display at least the second support information between the first support information and the second support information.
